(19) United States
(12) Patent Application Publication
(10) Pub. No.: US 2026/0212492 A1
(43) Pub. Date: Jul. 23, 2026

(54) DEEP CHANNEL ATTENTION MECHANISM FOR DIABETIC RETINOPATHY DETECTION

(71) Applicant: Ashish GHOSH, Kolkata (IN)

(72) Inventors: Ashish GHOSH, Kolkata (IN); Susmita GHOSH, Kolkata (IN); Abhiroop CHATTERJEE, Kolkata (IN)

(21) Appl. No.: 19/031,511

(22) Filed: Jan. 18, 2025

(51) Int. Cl. G06T 7/00; G16H 50/20

(52) U.S. Cl. CPC G06T 7/0012; G16H 50/20; G06T 2207/20084; G06T 2207/30041

(57) ABSTRACT

A method for detecting diabetic retinopathy (DR) integrates a deep channel attention mechanism (DCAM) into a pre-trained VGG19 convolutional neural network. The method fine-tunes the pre-trained VGG19 network with the deep channel attention module, which may leverage complementary information from various regions of fundus images to enhance the CNN's discriminative power. The DCAM incorporates an attention mechanism that selectively highlights salient features from images and fuses them with the original input. Simultaneous learning of attention weights for features and combination of attention-modulated features within the DCAM may allow the network to focus on relevant information while reducing the impact of noisy or irrelevant features. The method's performance has been evaluated on the APTOS DR Dataset.

### TECHNICAL FIELD

The invention relates to convolutional neural network (CNN) based image synthesis, and particularly relates to a methodology for enhancing the accuracy of diabetic retinopathy (DR) detection using deep learning technology by integrating a deep channel attention mechanism (DCAM) with a pre-trained VGG19 convolutional neural network.

### DESCRIPTION OF THE RELATED ART

In the field of diabetic retinopathy detection, various approaches have been explored using deep learning techniques. One common strategy involves the use of pre-trained convolutional neural networks (CNNs) to extract features from fundus images. These pre-trained models, such as VGG19, ResNet, and Inception, have proven effective in learning hierarchical representations from a wide range of images. However, the limitation of these approaches lies in their inability to selectively focus on the most relevant features within the fundus images. This can lead to suboptimal performance, as noisy or irrelevant information may influence the final diagnosis. Moreover, while attention mechanisms have gained popularity in deep learning, their integration into diabetic retinopathy detection models has been limited, leaving room for improvement in accuracy and specificity.

Another avenue explored in diabetic retinopathy detection is the use of traditional image processing techniques, such as feature engineering and segmentation algorithms. These methods rely on manually designed features, which may not capture the full spectrum of information present in fundus images. Additionally, they often require expert knowledge and are sensitive to variations in image quality and lighting conditions. As a result, these traditional approaches may not generalize well to diverse datasets and can be time-consuming to develop and maintain. With the increasing availability of large-scale diabetic retinopathy datasets, there is a growing demand for automated and data-driven solutions that can leverage the power of deep learning, attention mechanisms, and feature fusion to improve the accuracy and robustness of DR detection systems.

### SUMMARY OF THE INVENTION

The present invention introduces a novel methodology for the automated detection of diabetic retinopathy (DR) using deep learning techniques, with a specific focus on the integration of a deep channel attention mechanism (DCAM) into a pre-trained VGG19 architecture. The present invention improves the accuracy and discriminative power of DR detection systems for retinal fundus images.

The present invention integrates the deep channel attention module with a pre-trained VGG19 convolutional neural network (CNN) for more accurate DR detection. Here, the pre-trained VGG19 network is fine-tuned with the deep channel attention module. The module leverages the complementary information from various regions of fundus images to enhance the discriminative power of the CNN. The deep channel attention module incorporates an attention mechanism that selectively highlights salient features from images and fuses them with the original input. The simultaneous learning of attention weights for the features and thereupon the combination of attention-modulated features within the deep channel attention module facilitates the network's ability to focus on relevant information while reducing the impact of noisy or irrelevant features. The performance of the method is evaluated on a widely used dataset for DR classification e.g., the APTOS (Asia Pacific Tele-Ophthalmology Society) DR Dataset. The results confirm that the introduction of the fusion module (fusing of the deep channel attention module with CNN) improves the accuracy of DR detection as compared to the CNN alone. From the results, it is noticed that the attention mechanism could effectively capture the important visual cues from the fundus images.

### BRIEF DESCRIPTION OF DRAWINGS

An understanding of the present invention may be obtained by reference to the accompanying drawings, when taken in conjunction with the description herein and in which:

FIG. 1 illustrates a general representation of a deep neural network (DNN).

FIG. 2 illustrates a flow diagram of a deep channel attention mechanism (DCAM).

FIG. 3 illustrates different images taken from an APTOS, 2019 dataset.

FIG. 4 illustrates a flow diagram of a Deep Channel Attention Mechanism (DCAM) for Diabetic Retinopathy Detection.

FIG. 5 illustrates accuracy curves of a performance comparison between VGG19 and VGG19+DCAM.

FIG. 6 illustrates loss curves of a performance comparison between VGG19 and VGG19+DCAM.

FIG. 7 illustrates class predictions on unseen images with best results obtained using VGG19+DCAM.

FIG. 8 illustrates a confusion matrix for five different stages of diabetic retinopathy (DR) with the best result obtained using VGG19+DCAM.

### DETAILED DESCRIPTION OF PREFERRED EMBODIMENTS

Embodiments of the present invention are described below by way of example only. These examples represent the best ways of putting the invention into practice that are presently known to the Applicant although they are not the only ways in which this could be achieved. Forth the functions of the example and the sequence of steps for constructing and operating the example.

The present invention is directed to improve diabetic retinopathy (DR) detection, specifically focusing on the integration of a novel channel attention module with a pre-trained VGG19 Convolutional Neural Network (CNN).

In an embodiment, the present invention integrates a deep channel attention mechanism (DCAM) with a pre-trained CNN-based VGG19 model, enabling the network to selectively focus on salient features within fundus images and discard irrelevant information. By fine-tuning the pre-trained network with the channel attention block, the invention significantly improves DR detection accuracy when compared to the CNN alone. This has been validated using APTOS (Asia Pacific Tele-Ophthalmology Society) DR Dataset, demonstrating the ability of the present invention to effectively capture vital visual cues from fundus images, making it a valuable tool for automated DR diagnosis.

In an embodiment, the present invention among other components utilizes a VGG19 Convolutional Neural Network. The VGG19 network includes sixteen convolutional layers and three fully connected layers. In an exemplary embodiment, the architecture of VGG19 and dimensions of the feature maps after each layer when the input is an image with dimensions 224×224×3 (height×width×RGB channels) is illustrated below in Table 1:

TABLE 1

| Layers | Output dimensions |
|---|---|
| Convolutional Layer 1 with 64 filters of size 3 × 3, ‘same’ | 224 × 224 × 64 |
| padding, ReLU activation | |
| Convolutional Layer 2 (64 filters of size 3 × 3, ‘same’ | 224 × 224 × 64 |
| padding, ReLU activation | |
| Max-Pooling Layer 1 (2 × 2 pool size, stride 2) | 112 × 112 × 64 |
| Convolutional Layer 3 (128 filters of size 3 × 3, ‘same’ | 112 × 112 × 128 |
| padding, ReLU activation | |
| Convolutional Layer 4 (128 filters of size 3 × 3, ‘same’ | 112 × 112 × 128 |
| padding, ReLU activation | |
| Max-Pooling Layer 2 (2 × 2 pool size, stride 2) | 56 × 56 × 128 |
| Convolutional Layer 5 (256 filters of size 3 × 3, ‘same’ | 56 × 56 × 256 |
| padding, ReLU activation | |
| Convolutional Layer 6 (256 filters of size 3 × 3, ‘same’ | 56 × 56 × 256 |
| padding, ReLU activation | |
| Convolutional Layer 7 (256 filters of size 3 × 3, ‘same’ | 56 × 56 × 256 |
| padding, ReLU activation | |
| Convolutional Layer 8 (256 filters of size 3 × 3, ‘same’ | 56 × 56 × 256 |
| padding, ReLU activation | |
| Max-Pooling Layer 3 (2 × 2 pool size, stride 2) | 28 × 28 × 256 |
| Convolutional Layer 9 (512 filters of size 3 × 3, ‘same’ | 28 × 28 × 512 |
| padding, ReLU activation | |
| Convolutional Layer 10 (512 filters of size 3 × 3, ‘same’ | 28 × 28 × 512 |
| padding, ReLU activation | |
| Convolutional Layer 11 (512 filters of size 3 × 3, ‘same’ | 28 × 28 × 512 |
| padding, ReLU activation | |
| Convolutional Layer 12 (512 filters of size 3 × 3, ‘same’ | 28 × 28 × 512 |
| padding, ReLU activation | |
| Max-Pooling Layer 4 (2 × 2 pool size, stride 2) | 14 × 14 × 512 |

In an embodiment, the present invention provides a method for enhancing the accuracy of diabetic retinopathy (DR) detection by integrating a deep channel attention mechanism (DCAM) with a pre-trained VGG19 convolutional neural network (CNN). The key components implemented to carry out the desired method in accordance with the disclosure of the present invention are described below in a non-limiting way.

The deep channel attention mechanism (DCAM) is a critical component of the present invention. In an embodiment, the DCAM is configured to selectively amplify significant regions within retinal fundus images. The DCAM module enhances feature extraction within the CNN and aims to improve the CNN's ability to identify relevant information while reducing the impact of noisy or irrelevant features. The DCAM enhances the network's ability to focus on important details within retinal fundus images and stabilizes the network by some amount. The DCAM comprises several components, each contributing to the overall goal of improving feature extraction and enhancing the CNN's discriminative power.

In some embodiments, the DCAM is integrated with the pre-trained VGG19 architecture. The VGG19 is a deep neural network that may be pre-trained on a large dataset, for example, ImageNet. In the present invention, the VGG19 architecture is employed as a feature extractor, wherein it captures essential visual representations from the input data, for example, retinal fundus images.

In some embodiments, the DCAM implements a Weighted Global Average Pooling layer to compute weighted averages of input feature maps, effectively capturing the importance of each channel. Two back-to-back dense layers introduce non-linearity through a ReLU activation function and the final dense layer calculate attention weights through sigmoid activations, respectively. The computed attention weights are then applied to the input feature map through element-wise multiplication thereby giving priority to important regions and suppressing the less important ones. A skip connection preserves the extracted features from the last layer of VGG19 while incorporating attention-weighted features, resulting in an output with improved channel-wise emphasis.

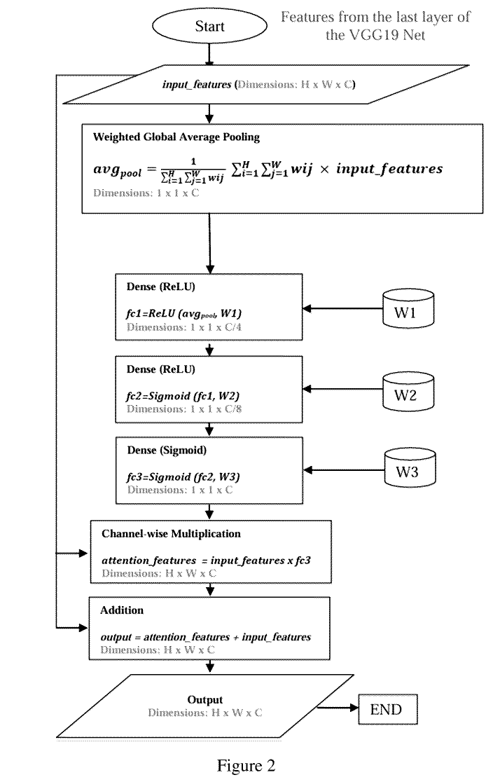

Weighted Global Average Pooling Layer: The Weighted Global Average Pooling layer is the initial layer applied to the features obtained from the pre-trained VGG19. It computes the weighted average of the feature maps along the spatial dimensions, effectively capturing the importance of each channel based on this weighted average. The output of this layer has reduced spatial dimensions (batch_size, 1, 1, C), wherein 1, 1 specify the dimensions of height and width of the output feature map and C represents the number of channels as mentioned in FIG. 2 of the present invention. It may be appreciated that during the training phase of deep neural networks, samples are trained in batches to reduce computation cost.

The Weighted Global Average Pooling layer is a custom layer that introduces weighted global average pooling to a neural network. It computes the average of the input feature map, and each channel is weighted differently based on the learnable weights. These weights are initialized to ones and can be trained during the network's training process. The layer multiplies each channel in the feature map by its corresponding weight, and the weighted sum across spatial dimensions (height and width) are computed for each channel. Finally, it normalizes the result by dividing by the sum of the learned weights, resulting in a weighted average representation of the input feature map. This mechanism allows the network to learn which channels are more informative and should contribute more to the final representation.

Dense Layers: The invention utilizes the inclusion of multiple dense layers within the DCAM. In a preferred embodiment, three dense layers, namely, a first dense layer, a second dense layer, and a third dense layer, are utilized. These three dense layers follow the Weighted Global Average Pooling layer. The first and second dense layers, with a rectified linear unit (ReLU) activation function, reduces the number of channels to C/ratio and C/(ratio*2) respectively, wherein the operator ‘/’ represents a division operation and ‘ratio’ denotes a tuneable parameter that the user can tune as per work and computation power requirements. The third dense layer employs a sigmoid activation function to produce an output feature with the same shape as the input of the first dense layer, which is (batch_size, 1, 1, C). These layers are implemented for their role in introducing non-linearity, channel dimension reduction, and the generation of attention weights for each channel.

First Dense Layer: The First layer utilizes a Rectified Linear Unit (ReLU) activation function and serves to reduce the number of channels to C/ratio, introducing non-linearity and compressing the channel dimension. The output from this layer has the shape (batch_size, 1, 1, C/ratio).

Second Dense Layer: The Second layer utilizes a Rectified Linear Unit (ReLU) activation function and serves to reduce the number of channels to C/(ratio*2), introducing non-linearity and compressing the channel dimension. The output from this layer has the shape (batch_size, 1, 1, C/(ratio*2)).

Third Dense Layer: The Third Dense layer employs a sigmoid activation function to produce an output with the same shape as the input of the first dense layer, which is (batch_size, 1, 1, C). The purpose of this layer is to determine the channel-wise importance by assigning attention weights to each channel.

Element-wise Multiplication: To incorporate the computed attention weights, the input feature vector is multiplied element-wise with the output feature vector from the third dense layer. This operation emphasizes important channel activations while maintaining the original spatial dimensions of the input feature. The resulting output has the shape (batch_size, height, width, C). The present invention claims the specific process of element-wise multiplication between the computed attention weights and the input feature. This step is essential for selectively emphasizing important channels while attenuating less relevant ones.

Skip Connection: To preserve the original information and facilitate gradient flow, a skip connection is employed. The attention feature vector obtained from the element-wise multiplication is added to the original input feature vector. This step forms a residual connection, ensuring that the original information is retained while incorporating channel attention. The output feature retains the same shape as the input feature (batch_size, height, width, C). The skip connection is vital for information flow and preservation of important details throughout the network. It does so by adding the attention feature vector to the original input feature map. The present invention utilizes the skip connection to add the attention feature obtained from the element-wise multiplication to the extracted input feature vector from the last layer of VGG19, forming a residual connection. This mechanism is critical for preserving the original information while incorporating channel attention.

After applying the channel attention mechanism, the output is flattened. Flattening means reshaping the multi-dimensional vector into a 1D vector, which is necessary when transitioning from convolutional layers to fully connected layers in many neural network architectures. Then a fully connected (Dense) layer with 1024 neurons and ReLU activation is added. This Dense layer connects every neuron from the previous layer to every neuron in this layer.

In the final step, another fully connected (Dense) layer is added with 5 neurons, which corresponds to the number of classes in the classification task. The activation function used here is softmax, which is typical for multi-class classification problems. It converts the raw scores (logits) from the previous layer into class probabilities. Each neuron in this layer represents the probability of the input belonging to a specific class.

The DCAM, including the Weighted Global Average Pooling and the element-wise multiplication with attention weights, effectively captures the importance of each channel in a feature map while preserving valuable channel-wise details. By calculating the weighted average activation of each channel across spatial dimensions, spatial information is condensed.

Subsequent operations involving dense layers with ReLU and sigmoid activations generate attention weights, providing a measure of relevance/importance for each channel. Further, these weights are applied to the feature map using element-wise multiplication, dynamically amplifying informative channels while attenuating less important ones. This selective emphasis on important channels enables the model to focus on relevant features and extract discriminative information effectively.

Transfer Learning: The transfer learning involves use of the pre-trained VGG19 model, which has been trained on the ImageNet dataset, as a feature extractor. This model captures powerful visual representations that are generalizable to various tasks. The channel attention module is integrated after the VGG19 layers, selectively emphasizing important channels in the extracted features. This combination of transfer learning and the attention module enhances the model's performance in diabetic retinopathy detection by improving feature extraction and the ability to focus on relevant information within retinal fundus images.

Image Preprocessing: The present invention utilizes the specific image preprocessing steps applied to retinal fundus images obtained from suitable datasets. In an embodiment, Retinal fundus images from APTOS 2019 dataset are categorized by severity level of diabetic retinopathy. In an embodiment, each image is resized to a fixed dimension of 224×224 pixels and the pixel values are normalized by dividing them by 255. This standardized preprocessing ensures consistent input data for the model.

Feature Extraction and Transfer Learning: The proposed method leverages the power of transfer learning by using pre-trained VGG19 models as feature extractors. These models have previously trained on the ImageNet dataset, capturing robust visual representations that are applicable to diverse tasks.

Discriminative DR Detection: The integration of the attention module with the VGG19 architecture significantly enhances the model's ability to discriminate between different DR severity levels. By selectively emphasizing important channels within the feature maps, the model can effectively extract relevant information from retinal fundus images, improving the accuracy of DR detection.

The present invention is the integration of the deep channel attention mechanism (DCAM) with the pre-trained VGG19 architecture to enhance the accuracy of diabetic retinopathy (DR) detection. The invention asserts its ability to effectively discriminate between different DR severity levels by selectively emphasizing important channels within the feature maps. This enhancement is claimed to result in a more accurate, reliable, and robust automated DR detection system compared to conventional CNN-based approaches.

The present invention claims the specific design and functionality of the deep channel attention mechanism (DCAM), image preprocessing steps, the utilization of transfer learning with pre-trained VGG19 models, and the overall enhancement of DR detection accuracy through the integration of these components. These claimed aspects collectively constitute the innovation and contributions of the invention in the field of diabetic retinopathy detection using deep learning.

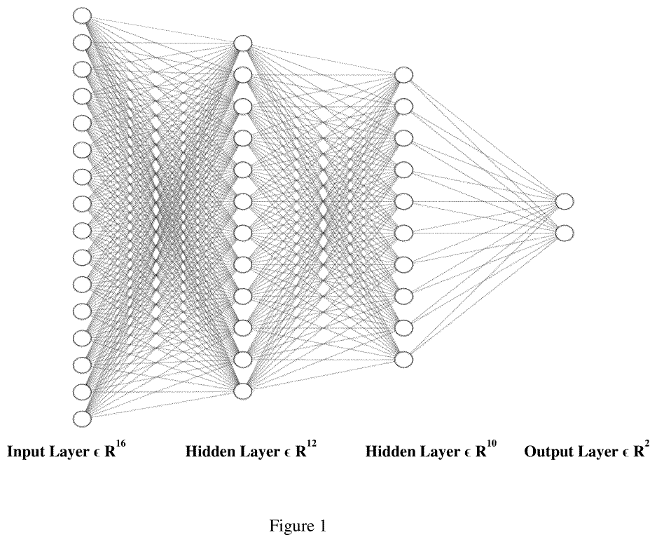

FIG. 1 illustrates a general representation of a deep neural network, wherein a schematic representation of a deep neural network (DNN) is provided. It typically consists of multiple layers, including an input layer, multiple hidden layers, and an output layer, with arrows or connections between them. This figure serves as a visual representation of the fundamental architecture of a DNN.

FIG. 2 illustrates a flow diagram of a deep channel attention mechanism (DCAM). The flow diagram illustrates the representation learning process within a deep neural network, particularly in the context of diabetic retinopathy detection.

FIG. 2 includes labeled components, such as Weighted Global Average Pooling, Dense layer1 with ReLU activation, Dense layer2 with ReLU activation, Dense layer3 with Sigmoid activation function, and element-wise multiplication. It demonstrates how these components are interconnected and integrated to improve feature extraction by selectively emphasizing important channel activations.

In an embodiment, the DCAM takes the input from the extracted features from the last Max-Pool layer of the VGG19 network. For an input feature map (14×14×512), the channel dimension is 512. When the Weighted Global Average Pooling is applied, the spatial dimensions reduces to 1×1×512. A fully connected dense layer1 with the channel dimension of 512 is divided by a constant kept at 4 so that 512/4=128 neurons are available in this layer. The output dimensions are (1,1,128). Further, a fully connected dense layer2 with the channel dimension of 512 is divided by a constant kept at 4*2=8 so that 512/8=64 neurons are available in this layer. The output dimensions are (1,1,64). Furthermore, another fully connected dense layer3 is applied with 512 neurons (same as the input channel dimension of DCAM). The output dimensions are (1,1,512). Finally, the element-wise multiplication is performed between the input feature map of DCAM and the output of the fully connected layer3. The output dimensions are (14,14,512) because the (1,1,512) vector is broadcasted to match the spatial dimensions of the input feature map. After applying the channel attention mechanism of 14×14×512 input feature map, the same spatial dimensions of 14×14×512 are still present, but the channel-wise information has been modulated based on the attention weights calculated by the network.

A skip connection between the input feature map obtained from the last layer of VGG19 and the output of the channel attention mechanism is provided as it may help to preserve important information and gradients during the training. The output dimensions after attention mechanism are (14,14,512). The output of the deep channel attention mechanism after element-wise multiplication is added to the original input feature map. The output dimensions after skip connection are (14,14,512). The skip connection ensures that the channel-attenuated feature map is combined with the original input feature map of the DCAM, preserving some of the original information while applying channel-wise attention. After applying the skip connection, the final output feature map has dimensions of (14,14,512), which is same as of the input feature map.

In an exemplary illustration, X is the input feature map with dimensions (H, W, C), where H is the height, W is the width, and C is the number of channels. A(X) is the output of the channel attention mechanism applied to X with dimensions (H, W, C) after attention. S(X) is the output feature map after adding the skip connection. The skip connection may be represented by Equation (1) as S(X)=X+A(X), where X and A(X) have dimensions (H, W, C), and the (+) operation denotes element-wise addition. Equation (1) essentially combines the original input feature map X with the attention-modulated feature map A(X) to produce the final output feature map S(X).

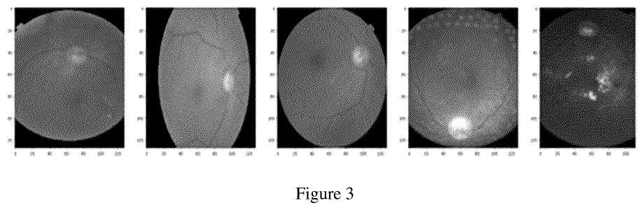

FIG. 3 illustrates different images taken from an APTOS, 2019 dataset which shows a set of retinal fundus images taken from the APTOS 2019 dataset. These images are used as input data for the diabetic retinopathy detection task. The images may vary in terms of quality, severity, and appearance, representing real-world data that the proposed methodology aims to analyze and classify.

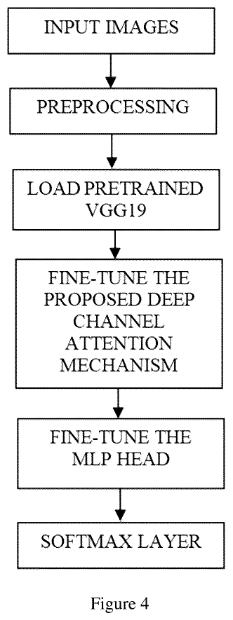

FIG. 4 illustrates a flow diagram explaining the process of a Deep Channel Attention Mechanism (DCAM) for Diabetic Retinopathy Detection. FIG. 4 provides the process explaining the methodology utilized for diabetic retinopathy detection. It includes steps such as data preprocessing, integration of the DCAM into a pre-trained VGG19 network, and the output layer for classification. The figure illustrates the sequential flow of data and operations within the methodology.

In Image preprocessing step, retinal fundus images are provided from a suitable dataset, for example, the APTOS 2019 dataset is utilized, which is categorized by the severity level of diabetic retinopathy. Each image is resized to a fixed dimension of 224×224 pixels. The pixel values are normalized by dividing them by 255 to ensure consistent input data for the implemented model.

Further, Feature Extraction and Transfer Learning is provided through a Block “Load pre-trained VGG19” architecture based model is provided, wherein the pre-trained VGG19 models trained on the ImageNet dataset are utilized as feature extractors. The VGG19 architecture captures powerful visual representations that are generalizable to various tasks.

A deep channel attention mechanism (DCAM) that includes components, i.e., Weighted Global Average Pooling Layer, Dense Layers, Element-wise Multiplication, and Skip Connection are provided, as illustrated in FIG. 2. Weighted Global Average Pooling Layer is provided to compute the weighted average of the feature maps along spatial dimensions, and captures the importance of each channel. Further, Dense Layers consist of multiple dense layers with ReLU and sigmoid activations to introduce non-linearity, channel dimension reduction, and generate attention weights for each channel. In Element-wise Multiplication, attention weights are applied to the input feature map through element-wise multiplication, emphasizing important channel activations. A skip connection is employed to preserve the original information and facilitate gradient flow during training.

An output layer through the Block “Fine-tune the Multi Layer Perceptron (MLP) Head” is provided, wherein after applying the DCAM, the output is flattened and passed through fully connected dense layer with ReLU activation.

Another fully connected dense layer with softmax activation is added, with 5 neurons corresponding to the number of classes in the classification task.

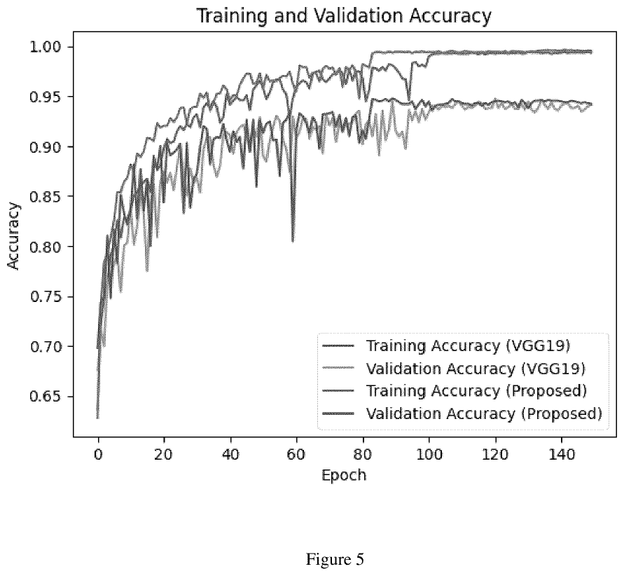

FIG. 5 illustrates accuracy curves of performance comparison between a VGG19 and a VGG19+DCAM block, wherein a comparison of the models' performance in terms of accuracy between the standard VGG19 architecture and the VGG19 architecture after incorporating the deep channel attention module (VGG19+DCAM). It illustrates accuracy curves that show how both the models perform over time or with varying conditions.

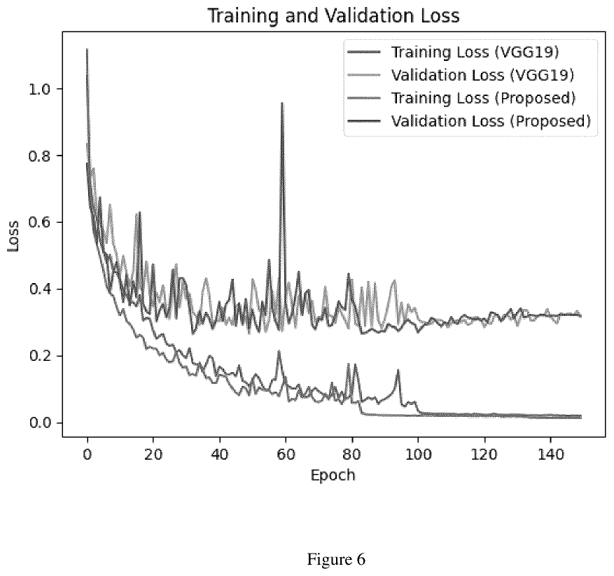

FIG. 6 illustrates loss curves of performance comparison between a VGG19 and a VGG19+DCAM block, wherein a comparison of the models' performance in terms of loss between the standard VGG19 architecture and the VGG19 architecture after incorporating the deep channel attention module (VGG19+DCAM). It illustrates how the models' loss values change throughout the training process, highlighting the potential advantages of the present channel attention module.

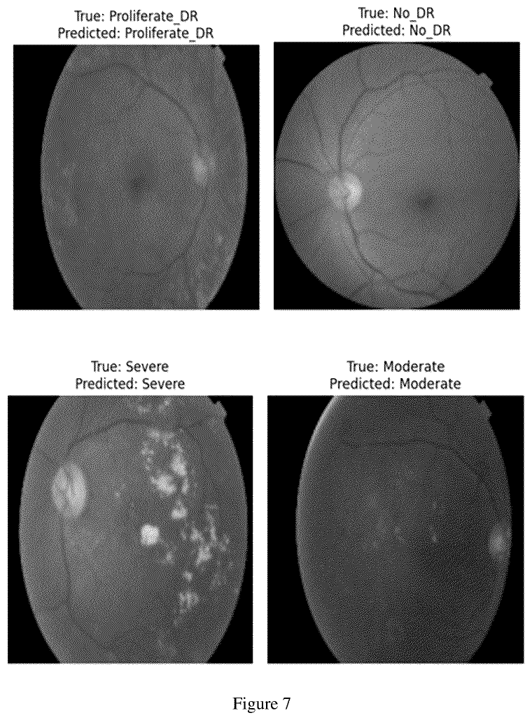

FIG. 7 illustrates class predictions on unseen images with the best results obtained using VGG19+DCAM of the present invention. FIG. 7 shows a set of retinal fundus images from an unseen dataset and displays the class predictions made by the methodology disclosed by the present invention. It demonstrates how well the model performs on new, previously unseen data. The figure may include the predicted class labels for each image.

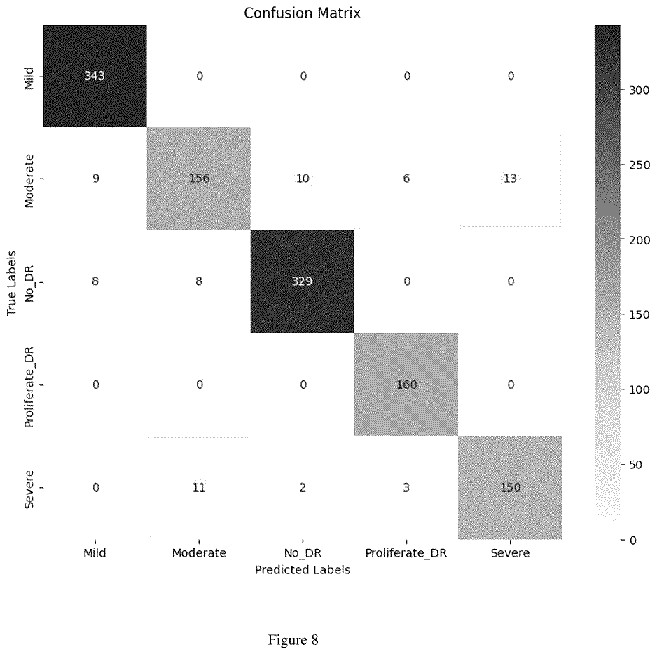

FIG. 8 illustrates a confusion matrix for five different stages of diabetic retinopathy (DR) with the best result obtained using VGG19+DCAM of the present invention. FIG. 8 presents a confusion matrix that summarizes the model's performance in classifying diabetic retinopathy into different stages. Each cell in the matrix represents the count or percentage of true positive, true negative, false positive, and false negative predictions for each severity level. It provides an in-depth evaluation of the model's classification accuracy for various DR stages.

In an embodiment, the present invention integrates a channel attention module with a pre-trained VGG19 CNN for diabetic retinopathy detection. The attention module enhances feature extraction, selective feature emphasis, and gradient flow, ultimately improving the model's accuracy in identifying different stages of diabetic retinopathy from retinal fundus images.

In the domain of convolutional neural networks (CNNs) for image processing and classification, several approaches have explored, ranging from custom-designed CNN architectures to the incorporation of Channel Attention Modules (DCAM) as disclosed in the present invention along with pre-trained models like VGG19.

In accordance with disclosure including FIGS. 2-8 of the present invention, a performance comparison of the methodology disclosed by the present invention with the existing techniques are provided, wherein an experimental setup including measures used for comparison and results are described. The analysis of results presented here provides a comprehensive evaluation of the proposed invention for diabetic retinopathy (DR) detection. It may be appreciated that the implemented experimental setup and the results produced thereby should not be construed to limit the scope of the present invention.

Experimental Setup: The experiments are conducted using an APTOS 2019 dataset, which contains high-resolution retinal fundus images and corresponding diagnostic labels. The experiments are performed on an NVIDIA A100 tensor core GPU. Twenty simulations were conducted to obtain reliable results.

Table 2 illustrates classification performance of the present invention as to the state-of-the-art models for the APTOS, 2019 dataset:

TABLE 2
Performance comparison of various deep neural networks

| Authors | Methods | Accuracy (%) |
|---|---|---|
| Dondeti et al. | Deep convolution features in non-linear | 77.90 |
| | embedding space | |
| Bodapati et al. | Blended multi-modal deep convnet features | 81.70 |
| Kassani et al. | Diabetic retinopathy classification using a | 83.09 |
| | modified Xception | |
| Liu et al. | Hybrid model structure for diabetic | 86.34 |
| | retinopathy classification | |
| Bodapati et al. | Composite deep neural network with gated- | 82.54 |
| | attention mechanism | |
| Sikder et al. | Classification of diabetic retinopathy using | 94.20 |
| | an ensemble learning | |
| Alyoubi et al. | DR fundus image classification and lesions | 89.00 |
| | localization | |
| Majumder et al. | MSEDenseNet | 81.00 |
| Podapati et al. | Feature Extraction using VGG16 and | 80.96 |
| | Xception | |
| Kassani et al. | Xception CNN | 83.09 |
| Rahman et al. | MobileNet V2 + SVM | 79.00 |
| Present invention | VGG19 + DCAM | 94.70 |

Table 2 summarizes the accuracy percentages achieved by different methods, including the present invention in the task of diabetic retinopathy (DR) detection. Each row corresponds to a specific method, and the accuracy percentage is reported in the “Accuracy (%)” column. The table presents a comparison of authors' methods and their corresponding accuracies in the context of diabetic retinopathy classification. Among the featured authors, Sikdar et al. achieved the highest accuracy (among all the state-of-the-art techniques) of 94.20% using an ensemble learning approach, showcasing the effectiveness of this technique while VGG19+DCAM has an edge and demonstrated notable performance with an accuracy of 94.70%. From Table 2, it may be observed that the accuracy percentages across the board vary from 77.90% to 94.70%, underscoring the diversity of approaches and the importance of selecting the most suitable method for accurate diabetic retinopathy classification.

Table 3 illustrates the performance comparison of accuracy of the present invention with different custom CNNs:

TABLE 3
Performance comparison of different custom CNNs

| Neural Network Architectures | Accuracy (%) |
|---|---|
| Custom CNN | 87.20 |
| CUSTOM CNN + DCAM | 89.00 |
| PRE-TRAINED VGG19 | 94.14 |
| PRE-TRAINED VGG19 + DCAM | 94.70 |

Notably, the Custom CNN achieved an accuracy rate of 87.20%, demonstrating the efficacy of tailoring CNNs to specific tasks. Furthermore, the combination of Custom CNN with DCAM led to an enhanced accuracy of 89.00%, highlighting the potential of attention mechanisms in improving model performance. Leveraging a pre-trained VGG19 model also yielded remarkable results, with an accuracy of 94.14%, highlighting the effectiveness of transfer learning in image classification tasks. However, the pre-trained VGG19+DCAM model outperformed them all with an impressive accuracy of 94.70%, emphasizing the significance of channel attention modules when coupled with established architectures for achieving state-of-the-art results in image analysis and classification endeavors. These findings underscore the continuous refinement and innovation in CNN-based methodologies for image-related tasks.

Table 4 illustrates the Accuracy values obtained with and without DCAM for VGG19 (Average of 20 simulations):

TABLE 4

| Methods | Accuracy (%) |
|---|---|
| VGG19 | 94.14 |
| VGG19 + DCAM | 94.70 (Average) |

Table 5 illustrates performance metrics on the APTOS 2019 dataset:

TABLE 5
Performance metrics on the APTOS 2019 dataset

| Metrics (Weighted Average) | VGG19 + DCAM (rounded) |
|---|---|
| Precision | 0.95 |
| Recall | 0.95 |
| F1-score | 0.95 |
| Accuracy (%) | 95 (Best) |
| Top-1 error (%) | 5 |

Table 4 compares the accuracy values obtained with and without the DCAM for the VGG19 model. The table shows that the VGG19+DCAM model achieved an accuracy of 94.70%, while the baseline VGG19 achieved an accuracy of 94.14%. This highlights that the incorporation of the channel attention module resulted in improved accuracy.

Accuracy Comparison: The invention reports that the proposed methodology, which includes the feature attention module integrated with VGG19 (denoted as VGG19+DCAM), achieved an accuracy of 94.70%. This is a promising result as accuracy is a crucial metric in medical image analysis, especially for DR detection.

Performance Metrics: The table labeled Table 5 provides a summary of various performance metrics for the VGG19+DCAM model. These metrics include Precision, Recall, F1-score, Accuracy, and Top-1 error. All of these metrics show high values, indicating the effectiveness of the proposed method.

Precision: The precision score of 0.95 indicates that the model has a high ratio of true positive predictions to the total predicted positive instances.

Recall: The recall score of 0.95 suggests that the model can effectively identify a high percentage of actual positive instances.

F1-Score: The F1-score of 0.95 is a balanced measure that takes both precision and recall into account, and it also indicates strong performance.

Accuracy: The accuracy of 95.2% (highest across 20 runs) is a crucial measure in classification tasks, indicating the proportion of correct predictions made by the model.

Top-1 Error: The low top-1 error of 4.8% further confirms the accuracy and effectiveness of the proposed model.

The present invention concludes by summarizing the key findings. It highlights that the integration of the channel attention module enhances the model's accuracy in identifying different severity levels of DR by emphasizing more to the important channels while deemphasizing the less important ones. The methodology combines transfer learning from VGG19 with the channel attention block, showcasing its potential for early detection and diagnosis of DR.

The analysis of results presented here demonstrates that the proposed framework, augmented with the channel attention module, significantly improves the accuracy and effectiveness of diabetic retinopathy detection compared to both baseline models and state-of-the-art methods. These results are promising for the early diagnosis of DR, which is crucial for effective management and intervention.

In accordance with the disclosure of the present invention, the present invention provides multi-fold advantages over the existing previous channel attention mechanisms such as Squeeze and Excitation Net, SENet, etc. For instance, SENet's channel attention mechanism aims to capture global information from the entire feature map to learn channel-wise scaling factors. In contrast, instead of explicitly calculating global statistics, the present invention utilizes weighted global average pooling which allows the network to capture global information with a lightweight operation.

Moreover, SENet provides complex modeling wherein multiple layers and operations are employed to capture complex relationships between channels. It explicitly calculates global statistics through global average pooling and uses these statistics to model channel dependencies. In contrast, the present invention implements a simple and efficient custom channel attention mechanism wherein channel-wise scaling factors are computed directly from the weighted global average pooling layer, and these factors are obtained by applying a dense layer with ReLU activations. The channel-wise scaling factors are further passed through a sigmoid activation, ensuring that they range from 0 to 1.

The present invention provides advantage of preserving original features. The distinctive feature of the custom mechanism is the inclusion of a residual connection. After computing the channel-wise scaling, we add the extracted input features obtained from the last layer of VGG19 back to the attention-modulated features.

The present invention provides advantage of preservation of information. This residual connection is intended to help preserve critical information from the extracted feature map (from the last layer of VGG19) while benefiting from the learned attention weights.

The custom channel attention mechanism of the present invention prioritizes simplicity and computational efficiency while still allowing the network to adaptively scale individual channels. The key innovation is the introduction of a residual connection, which can help maintain crucial information from the extracted feature map received from the last layer of VGG19.

The present invention suggests future work that may involve applying the disclosed method to larger and more diverse datasets, indicating avenues for further research and improvement.

In accordance with the disclosure of the present invention, a computer platform or computing unit may be provided for detecting diabetic retinopathy, wherein plurality of hardware components are required for training a model to integrate the deep channel attention mechanism (DCAM) into a pre-trained VGG19 Convolutional Neural Network (CNN). The computing unit comprises a number of hardware units and components, which is configured to train the models to perform the desired functions as claimed in the present invention. The hardware units and components comprises plurality of processing units including Graphics Processing Unit (GPU), Central Processing Unit (CPU), etc., memory and storages, and communication modes for connecting with remote computing units, such as cloud servers, etc. In some embodiments, the GPU is Google Colab with a V100 GPU. The V100 GPU is a powerful GPU designed by NVIDIA and belongs to the Volta architecture. It is well-suited for deep learning tasks and is commonly used for accelerating neural network training. The CPU is responsible for general purpose computing tasks. During the training of deep learning models, the CPU is involved in data pre-processing, whereas training is performed on GPUs utilizing their parallel processing power. In some embodiments, the Memory is a Random Access Memory (RAM) that is essential for storing and quickly accessing data during model training. As sufficient RAM is crucial, especially when dealing with large datasets and complex models. In Google Colab, the amount of available RAM (83 GB) depends on the type of virtual machine is provided. Storage is necessary for storing datasets, model checkpoints, and other files related to the training process. Google Colab provides cloud storage. Internet Connectivity is required for data transmission for which a stable internet connection is required for accessing and downloading datasets, libraries, and other resources during the development and training process. For cloud-based platforms like Google Colab, a reliable internet connection is essential. For deployment, network connectivity is necessary for communication between the deployed model and the application or service using it. In some embodiments, a server deployed suitable specifications for serving the trained model. The server may include processing units such as CPU, GPU, etc., of required capacity, memory such as RAM, and storage.

While this invention has been described in connection with what is presently considered to be the most practical and preferred embodiment, it is to be understood that the invention is not limited to the disclosed embodiments, but, on the contrary, is intended to cover various modifications and equivalent arrangements included within the scope of the appended claims.

We claim:

1. A method for detecting diabetic retinopathy by integrating a deep channel attention mechanism (DCAM) into a pre-trained VGG19 Convolutional Neural Network (CNN), comprising:
receiving features from the pre-trained VGG19 Convolutional Neural Network (CNN);
computing weighted averages of one or more input feature maps along the spatial dimensions thereof by a weighted global average pooling layer and generating an input feature vector with reduced spatial dimensions, wherein the weighted averages of the input feature maps are computed using learnable weights of each channel;
providing at least three dense layers in the DCAM, the layers comprising:
a first dense layer with a rectified linear unit (ReLU) activation function configured to reduce the number of channels to C/ratio;
a second dense layer with a rectified liner unit (ReLU) activation function configured to reduce the number of channels to C/(ratio*2); and
a third dense layer with a sigmoid activation function to generate an output vector, wherein dimensions of the output vector of the third dense layer is (batch_size, 1, 1, C), wherein C is the channel and ratio is a tuneable parameter,
generating an attention feature vector by multiplying the weighted averages and the input feature vector, wherein the multiplication is an element-wise multiplication;
adding the attention feature vector obtained from the element-wise multiplication and the extracted input feature vector from the last layer of the pre-trained VGG19 architecture to generate a residual connection, wherein a skip connection is utilized for adding the said features;
providing a fully connected dense layer with the ReLU activation function, the dense layer connects every neuron from the previous layer to every neuron available therein; and
providing a last fully connected dense layer with 5 neurons corresponding to the number of classes in a classification task.

2. The method of claim 1 wherein the dimensions of the output vector of the third dense layer and the first dense layer are same.

3. The method of claim 1 further comprising the step of image preprocessing applied to plurality of retinal fundus images obtained from a dataset.

4. The method of claim 1 wherein the activation function is a softmax activation function, the softmax activation function converts the raw scores from the previous layer into class probabilities.

5. The method of claim 1 wherein the VGG19 Convolutional Neural Network is pre-trained on an APTOS DR Dataset.
